(19) United States
(12) Patent Application Publication
(10) Pub. No.: US 2023/0233600 A1
(43) Pub. Date: Jul. 27, 2023

(54) IMPROVED VIRUCIDAL FORMULATIONS

(71) Applicant: FIREBRICK PHARMA LIMITED, Melbourne (AU)

(72) Inventors: Peter MOLLOY, Creswick (AU); Simon TUCKER, Lysterfield (AU); Stephen GOODALL, Blackwall (AU)

(73) Assignee: FIREBRICK PHARMA LIMITED, Melbourne (AU)

(21) Appl. No.: 18/001,343

(22) PCT Filed: Jun. 10, 2021
(86) PCT No.: PCT/AU2021/050590

§ 371 Date: Dec. 9, 2022

(30) Foreign Application Priority Data
Jun. 10, 2020 (AU) 2020901910

(51) Int. Cl. A61K 33/18; A61K 47/10; A61K 47/32; A61P 31/16; A61P 31/14

(52) U.S. Cl. CPC A61K 33/18; A61K 47/10; A61K 47/32; A61P 31/16; A61P 31/14

(57) ABSTRACT

The present invention is directed to PVP-I formulations having enhanced virucidal activity. The formulations are intended for topical administration for treatment and/or decreased risk of microbial infections in subjects. The formulations include PVP-I and other ingredients selected to enhance the virucidal activity of the formulation over PVP-I alone.

### FIELD

The present invention is directed to povidone-iodine (PVP-I) formulations having enhanced virucidal activity. The formulations are intended for topical administration for treatment and/or decreased risk of viral infections in subjects. The formulations include PVP-I and other ingredients selected to enhance the virucidal activity of the formulation over PVP-I alone.

### BACKGROUND

Povidone-iodine (PVP-I) is a complex of polyvinylpyrrolidone and an effective concentration of iodine (generally 9-12%). PVP-I is considered to have a broader spectrum of antimicrobial action compared with other common antiseptics such as chlorhexidine, octenidine, polyhexanide 20 and hexetidine 26 with reported ability to inactivate Gram-positive and Gram-negative bacteria, bacteria spores, fungi, protozoa and enveloped and non-enveloped viruses (Lachapelle J, et al. Antiseptics in the era of bacterial resistance: a focus on povidone iodine. Future Med 2013; 10: 579-92; Gocke D J, et al. In vitro studies of the killing of clinical isolates by povidone-iodine solutions. J Hosp Infect. 1985 March; 6 Suppl A:59-66; Berkelman R L, et al. Increased bactericidal activity of dilute preparations of povidone iodine solutions. Journal of clinical microbiology 1982; 15:635-9; Eggers M. Infectious Disease Management and Control with Povidone Iodine. Infect Dis Ther. 2019 December; 8(4):581-593; Sriwilaijaroen N. et al. Mechanisms of the action of povidone-iodine against human and avian influenza A viruses: its effects on hemagglutination and sialidase activities. Virol J. 2009 Aug. 13; 6:124; Eggers M, et al. Rapid and Effective Virucidal Activity of Povidone-Iodine Products Against Middle East Respiratory Syndrome Coronavirus (MERS-CoV) and Modified Vaccinia Virus Ankara (MVA). Infectious diseases and therapy 2015; 4:491-501; Kawana R, et al. Inactivation of human viruses by povidone iodine in comparison with other antiseptics. Dermatology 1997; 195 Suppl:29-35; 20 Kariwa H et al. Inactivation of SARS coronavirus by means of povidone iodine, physical conditions and chemical reagents. Dermatology 2006; 212 Suppl:119-23).

PVP-I is routinely used as a general antiseptic in ophthalmology and surgery and there have been numerous clinical studies demonstrating the safety of PVP-I in a variety of topical applications in ophthalmology, otology, rhinology and dermatology. PVP-I in nasal usage has been explored for nasal decolonization of potentially pathogenic bacteria (see for example Rezapoor M, et al. Povidone-Iodine-Based Solutions for Decolonization of Nasal Staphylococcus aureus: A Randomized, Prospective, Placebo-Controlled Study. The Journal of Arthroplasty. 2017; 32:9:2815-19) the treatment and prevention of the common cold (Molloy and Goodall, Australian patent 2014206143) and recently it has been proposed as an intervention to assist in the management of SARS-CoV-2 (see for example WO 2020/232515 and U.S. Ser. No. 16/925,740).

Povidone-iodine or PVP-I is a broad-spectrum topical microbicide that is known to rapidly inactivate a wide range of micro-organisms, principally through the potent oxidative effects of free iodine on microbial proteins and nucleic acids. Because these effects are potent and non-specific, PVP-I has shown no susceptibility to antimicrobial resistance development despite more than 30 years of extensive usage as a topical antiseptic. More recently, it has found applicability as a microbicide for use on mucous membranes.

Low PVP-I concentrations, in the range of 0.1% to 1.25% w/v are known to be generally safe for use on mucous membranes, such as in the nasal passages, where infections by microorganisms, notably viruses, are common. However, the dose form and other components of the formulation which comprise a pharmaceutically acceptable medication having enhanced virucidal activity are desirable.

The most common infection of the nasal passages is the common cold, which is usually caused by viruses and is otherwise referred to as an acute upper respiratory tract infection or URI. The most common viral pathogen that causes acute URIs is human rhinovirus (HRV). The use of PVP-I at low concentrations for the treatment and prevention of the common cold caused by HRV and other viruses has been previously disclosed.

In addition to URIs, viruses can cause other relevant problems in the nasal passages. Notably, highly pathogenic viruses, such as pandemic influenza, SARS-CoV-2 and similar viruses, can produce asymptomatic or mild infections in the nasal passages, using the nasal passages as a site of replication and transmission, rather than necessarily causing a manifest clinical infection in the nose. This is relevant because the proliferating virus can travel to other sites in the respiratory tract, such as the lungs, and cause more serious disease, or it can be shed from the nasal passages to infect other people. The use of low concentrations of PVP-I in the nasal passages for the prevention of transmission of such highly pathogenic viruses has been disclosed (see for example WO 2020/232515, US20200384016).

### SUMMARY OF THE INVENTION

The present inventors have developed formulations containing PVP-I which have enhanced virucidal activity. This enhanced activity is particularly surprising as the enhanced activity was only seen against viruses and not bacteria. The present invention therefore provides a PVP-I containing formulations with specific enhanced activity which has particular therapeutic use. The present invention relates to effective combinations of concentrations of PVP-I no greater than 1.25% together with other active constituents in a fixed dose combination product composition that is safe and can be readily administered via a suitable intranasal spray, or similar device. The formulations can be used in the management of viral infections of the respiratory tract and/or viral infections that utilise the respiratory tract as a portal of entry and/or dissemination.

In one aspect the present invention provides an aqueous virucidal formulation (based on % w/v of the total formulation) comprising:
a) about 0.10% to about 1.25% w/v PVP-I;
b) 0.00% to about 15% w/v of hyaluronic acid, polyethylene glycol and/or glycerol;
c) 0.00% to about 0.20% w/v of menthol;
d) 0.00% to about 30% of a polar solvent;
e) 0.00% to about 5% of sodium hydrogen phosphate or similar buffer; and
f) 0.00% to about 0.20% w/v of iodide and/or iodate salt;

wherein the formulation comprises at least two of (b)-(f).

In a preferred embodiment of the present invention the aqueous virucidal formulation comprises:

about 0.10% to about 1.25% w/v PVP-I;

about 0.005% to about 0.20% w/v of iodide and/or iodate salt; and

about 0.005% to about 0.05% w/v menthol.

It is further preferred that the aqueous virucidal formulation comprises:

about 0.10% to about 1.25% w/v PVP-I;

about 0.005% to about 0.01% w/v of an iodate salt;

about 0.005% to about 0.05% w/v menthol;

about 0.005% to about 0.10% w/v of an iodide salt; and

about 1.0% w/v to about 15% w/v of glycerol.

It is further preferred that the aqueous virucidal formulation comprises:

about 0.10% to about 1.25% w/v PVP-I;

about 0.005% to about 0.01% w/v of an iodate salt;

about 0.005% to about 0.05% w/v menthol;

about 0.005% to about 0.10% w/v of an iodide salt;

about 1.0% w/v to about 15% w/v of glycerol; and

about 0.2% w/v to about 30% w/v of a polar solvent other than water.

It is still further preferred that the aqueous virucidal formulation comprises:

about 0.10% to about 1.0% w/v PVP-I;

about 0.005% to about 0.01% w/v of an iodate salt;

about 0.005% to about 0.05% w/v menthol;

about 0.005% to about 0.10% w/v of an iodide salt;

about 1.0% w/v to about 10% w/v of glycerol;

about 0.2% w/v to about 1.0% w/v of ethanol;

about 0.05% to about 0.20% w/v of sodium dihydrogen phosphate dihydrate;

sodium hydroxide q.s.; and

water q.s.

A preferred concentration range of PVP-I is about 0.25% to about 1.00%, preferably about 0.50%.

The aqueous virucidal formulation preferably has a pH of about 3 to about 6.

In another aspect the invention provides an aqueous virucidal formulation (based on % w/v of the total formulation) comprising:
(a) about 0.10% to about 1.25% w/v PVP-I;
(b) 0.00% to about 15% w/v of hyaluronic acid, polyethylene glycol and/or glycerol;
(c) 0.00% to about 0.20% w/v of menthol;
(d) 0.00% to about 30% of a polar solvent;
(e) 0.00% to about 5% of sodium hydrogen phosphate or similar buffer; and
(f) 0.00% to about 0.20% w/v of iodide and/or iodate salt;
wherein the formulation comprises at least two of (b)-(f).

### BRIEF DESCRIPTION OF THE DRAWINGS

FIG. 1 shows a comparison of the published time dependent virucidal activity at clinically relevant exposure times of formulations containing 0.45-1.0% PVP-I against HRV-14, compared with representative inventive formulation (Formulation 2) containing 0.5% PVP-I. Units are log 10 viral titre reduction. The heavy horizontal line corresponds to a 4 log 10 reduction (corresponding to a 99.99% reduction in viral infectivity) in accordance with the Therapeutic Goods Administration (TGA) guidelines for virucidal testing: “TGA instructions for disinfectant testing, Disinfectants, sterilants and sanitary products”, Version 2.1, March 2020 (TGA Department of Health, Australian Government).

### REFERENCE FORMULATIONS TESTED WERE

1 Isodine 1.0% PVP-I: Data from Kawana et al. Dermatology 1997, 195:29-35
2 Betaisodona 0.5% PVP-I: Data from Wutzler et al. Antiviral Research 2002, 54:89-97
3 Betaisodona 0.5% PVP-I: Data from Reimer et al. Dermatology 2002, 204:114-120
4 Liposomal 0.45% PVP-I: Data from Wutzler et al. Antiviral Research 2002, 54:89-97
5 PVP-I 1.0% in phosphate buffer: Data from Kawana et al. Dermatology 1997, 195:29-35

### Definitions

As used herein, “free iodine” refers to that elemental or diatomic iodine which is in solution and not actually bound to the polymer, in the case of povidone-iodine, although it may initially have been so bound. The free iodine concentration represents the instantaneous microbicidal potency of the iodophor solution and is measured according to methods taught in U.S. Pat. No. 3,028,300 to Cantor, incorporated herein by this reference.

As used herein, “available iodine” refers to that iodine of the iodophor which is ultimately available to be released from the polymer as free iodine. It therefore includes free iodine in solution, diatomic iodine available from tri-iodide ions as well as diatomic iodine held within a reservoir formed by the polymer structure. The available iodine does not include iodide ions. Available iodine is measured by thio sulfate titration in accordance with United States and British Pharmacopeia monographs.

As used herein, “total iodine” refers to all forms of iodine including free iodine, available iodine, iodide, iodate and other charged species of iodine in solution.

As used herein, “effective amount” refers to the dosage volume and frequency of the administration of the aqueous formulation according to the inventive method, which is sufficient to be effective in the application. The effective amount will vary in a manner which would be understood by a person of skill in the art with patient age, sex, weight, nasal passage volume etc. An appropriate dosage and dosage frequency can be ascertained through routine trial.

As used herein “polar solvent” means a protic solvent other than water that contains a labile H ion.

As used herein, “ambient temperature” refers to the temperature in the environment at which the method of the current invention is conducted in one of the preferred embodiments. Typically ambient temperature will be about 10° C. to about 30° C. Importantly the term “ambient temperature” means that neither the formulation nor the nasal passages of the subject to be treated are exposed to external heating in carrying out the method of the present invention.

### DETAILED DESCRIPTION OF THE INVENTION

The present inventors have shown that virucidal formulations comprising PVP-I can be surprisingly improved (either in a supra-additive or synergistic manner) by combining with certain know pharmaceutically acceptable ingredients.

The aqueous formulations of the present invention may be presented in the form a solution, drops, spray, gel, cream, aerosol, or inhalant.

The formulations disclosed herein are aqueous formulations and as such contains water as the major carrier/diluent. In certain embodiments water makes up from about 70% w/v to about 96% w/v of the total formulation, such as about 75% w/v, 78% w/v, 80% w/v, 82% w/v, 84% w/v, 86% w/v, 88% w/v, 90% w/v, 92% w/v, 94% w/v, 96% w/v, or about 97% w/v, or any range between such concentrations. The other formulation ingredients are typically first added together and water is then added QS (quantum satis) to make up 100% w/v of the total composition.

The formulations disclosed herein comprise about 0.10% to about 1.25% w/v PVP-I of the total formulation. PVP-I (also known as povidone-iodine and iodopovidone) may be presented within the formulation as liposomal PVP-I or non-liposomal PVP-I.

Suitable concentration of PVP-I is between 0.10% w/v and 1.25% w/v, such as about 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, or about 1.2% or any range within such concentrations.

The formulations also comprise quantities of iodide and/or iodate salt (0.005% to about 0.20% w/v), and preferably potassium iodide and/or potassium iodate.

In certain embodiments the formulations comprise 0.005% to about 0.10% w/v potassium iodide of the total formulation.

Suitable concentration of potassium iodide is between 0.005% w/v and 0.10% w/v, such as about 0.006%, 0.007%, 0.008%, 0.009%, 0.01%, 0.02%, 0.03%, 0.04%, 0.05%, 0.06%, 0.07%, 0.08%, or about 0.09% w/v or any range within such concentrations.

In certain embodiments the formulations comprise about 0.001% to about 0.01% w/v potassium iodate of the total formulation.

Suitable concentration of potassium iodate is between 0.001% w/v and 0.01% w/v, such as about 0.002%, 0.003%, 0.004%, 0.005%, 0.006%, 0.007%, 0.008%, or about 0.009% w/v or any range within such concentrations.

In certain aspects the formulations may also comprise about 0.005% to about 0.20% w/v menthol of the total formulation.

Suitable concentration of menthol is between 0.005% w/v and 0.20% w/v, such as about 0.006%, 0.007%, 0.008%, 0.009%, 0.01%, 0.02%, 0.03%, 0.04%, 0.05%, 0.06%, 0.07%, 0.08%, 0.09%, 1.0%, 1.5%, 1.8% or about 1.9% w/v or any range within such concentrations.

In certain aspects the formulations may also comprise about 1.0% to about 15% w/v hyaluronic acid, polyethylene glycol and/or glycerol of the total formulation, preferably glycerol.

In certain embodiments the formulations disclosed herein additionally includes an amount of glycerol (glycerine) from about 1 to about 15% w/v of the total preparation. In certain embodiments the formulations disclosed herein additionally includes an amount of glycerol (glycerine) from about 1 to about 10% w/v of the total preparation.

Suitable concentration of hyaluronic acid, polyethylene glycol and/or glycerol is between 1.0% w/v and 15% w/v, such as about 2%, 3%, 4%, 5%, 6%, 7%, 8%, 9%, 10%, 11%, 12%, 13%, or about 14% w/v or any range within such concentrations.

In certain embodiments the formulations disclosed herein may also include one or more of the following: solubilising agents, polar solvents, dry acidulant, sequestrants, alkaline agents, counter-irritant agents, local anaesthetic and/or preservatives.

In certain aspects the formulations may also comprise an amount of a polar solvent, such as isopropyl alcohol and ethanol, from about 0.2 to about 30% w/v of the total preparation.

Suitable concentrations of the polar solvent is between 0.2% w/v and 30% w/v, such as about 0.4%, 0.5%, 0.6%, 0.7%, 0.8%, 0.9%, 1.0%, 1.2%, 1.3%, 1.4%, 1.5%, 1.6%, 1.7%, 1.8%, 1.9%, 2.5%, 5%, 10%, 15%, 20% or about 25% w/v or any range within such concentrations.

In certain embodiments the formulations are aqueous based intranasal preparations where water makes up from about 70% w/v to about 96% w/v of the total preparation and has a pH of from about 2-7 such as a pH range of about 3-6, such as about 2, 3, 4, 5 or 6, or any range within.

Accordingly in order to adjust the pH certain embodiments the formulations disclosed herein additionally includes an amount of an alkaline agent, such as sodium hydroxide, from about 0.01 to about 2% w/v of the total preparation.

Suitable concentrations of the alkaline agent is between 0.01% w/v and 2% w/v, such as about 0.03%, 0.05%, 0.07%, 0.09%. 0.1%, 0.2%, 0.3%, 0.4%, 0.5%, 0.6%, 0.7%, 0.8%, 0.9%, 1.0%, 1.2%, 1.3%, 1.4%, 1.5%, 1.6%, 1.7%, 1.8%, or about 1.9% w/v or any range within such concentrations.

In certain embodiments the formulations disclosed herein optionally includes an amount of a preservative, such as a quaternary ammonium salt preservative, from about 0.01 to about 0.5% w/v of the total preparation.

Suitable concentrations of the preservative is between 0.01% w/v and 0.5% w/v, such as about 0.03%, 0.05%, 0.07%, 0.09%. 0.1%, 0.2%, 0.3%, or about 0.4% w/v or any range within such concentrations.

In certain embodiments the formulations disclosed herein additionally includes an amount of a buffer such as sodium phosphate, citrate or acetate from about 0.05 to about 0.5% w/v of the total preparation, such as 0.06%, 0.07%, 0.08%, 0.09%, 0.1%, 0.2%, 0.3%, or about 0.4% w/v or any range within such concentrations.

The present invention comprises methods for the treatment and prevention antimicrobial infections the methods in all cases employing application to the nasal passages of human subjects of the aqueous formulation at ambient temperature. The inventive methods produce substantial positive outcomes in key clinical measures of the infections, including reducing the severity of symptoms and the duration of the infection and thereby constitute methods for the effective treatment of a viral infections known to be treatable with PVP-I, such as those discussed in Australian Patent No. 20144206143 and WO 2020/232515 which are incorporated here in their entirety.

In another preferred embodiment the pharmaceutical preparation is administered into the nostrils of the human subject at least once, and preferably between 1 and 12 times, daily with between about 50 μL and about 1000 μL of the pharmaceutical preparation administered to each nostril for each administration of the preparation.

In another preferred embodiment the pharmaceutical preparation is in a dosage form selected from the group consisting of intranasal solutions, drops, sprays, gels, aerosols, or inhalants, but may include any other device or formulation suitable for administering an effective amount of the formulation to the nasal passages.

In order that the nature of the present invention may be more clearly understood preferred forms thereof will now be described with reference to the following non-limiting examples.

### EXAMPLES

Exemplary Aqueous PVP-I Formulations

### Formulation 1:

| Per 100 mL | g | % w/v |
|---|---|---|
| PVP-I | 0.80 | 0.80% |
| Potassium Iodide | 0.010 | 0.010% |
| Potassium Iodate | 0.005 | 0.005% |
| Glycerol | 4.80 | 4.80% |
| Sodium DiHydrogen | 0.15 | 0.15% |
| Phosphate Dihydrate | | |
| Sodium Hydroxide | 0.75 | 0.75% |
| Ethanol | 0.49 | 0.49% |
| Menthol | 0.01 | 0.010% |
| Benzalkonium Chloride | 0.1 | 0.10% |
| Water | 93.08 | 93.08% |
| | 100.00 | 100.0% |

### Formulation 2:

| Per 100 mL | g | % w/v |
|---|---|---|
| PVP-I | 0.50 | 0.50% |
| Potassium Iodide | 0.050 | 0.050% |
| Potassium Iodate | 0.005 | 0.005% |
| Glycerol | 5.00 | 5.00% |
| Sodium DiHydrogen | 0.15 | 0.15% |
| Phosphate Dihydrate | | |
| Sodium Hydroxide | 0.75 | 0.75% |
| Ethanol | 0.50 | 0.50% |
| Menthol | 0.01 | 0.010% |
| Water | 93.035 | 93.035% |
| | 100.00 | 100.0% |

### Formulation 3:

| Per 100 mL | g | % w/v |
|---|---|---|
| PVP-I | 1.00 | 1.00% |
| Potassium Iodide | 0.010 | 0.010% |
| Potassium Iodate | 0.005 | 0.005% |
| Glycerol | 5.50 | 5.50% |
| Sodium DiHydrogen | 0.15 | 0.15% |
| Phosphate Dihydrate | | |
| Sodium Hydroxide | 0.75 | 0.75% |
| Ethanol | 0.49 | 0.49% |
| Menthol | 0.01 | 0.010% |
| Water | 93.08 | 93.08% |
| | 100.00 | 100.0% |

### Formulation 4:

| Per 100 mL | g | % w/v |
|---|---|---|
| PVP-I | 1.25 | 1.25% |
| Potassium Iodide | 0.010 | 0.010% |
| Potassium Iodate | 0.005 | 0.005% |
| Glycerol | 4.60 | 4.40% |
| Sodium DiHydrogen | 0.325 | 0.325% |
| Phosphate Dihydrate | | |
| Sodium Hydroxide | 0.25 | 0.25% |
| Ethanol | 0.22 | 0.22% |
| Menthol | 0.21 | 0.21% |
| Benzalkonium Chloride | 0.25% | 0.25% |
| Water | 93.08 | 93.08% |
| | 100.00 | 100.0% |

### Formulation 5:

| Per 100 mL | g | % w/v |
|---|---|---|
| PVP-I | 0.30% | 0.30% |
| Potassium Iodide | 0.010 | 0.010% |
| Potassium Iodate | 0.005 | 0.005% |
| Glycerol | 5.20 | 5.20% |
| Sodium DiHydrogen | 0.35 | 0.35% |
| Phosphate Dihydrate | | |
| Sodium Hydroxide | 0.55 | 0.55% |
| Ethanol | 0.49 | 0.49% |
| Menthol | 0.01 | 0.010% |
| Benzalkonium Chloride | 0.08 | 0.08% |
| Water | 93.0 | 93.0% |
| | 100.00 | 100.0% |

### Formulation 6:

| Per 100 mL | g | % w/v |
|---|---|---|
| PVP-I | 0.10 | 0.10% |
| Potassium Iodide | 0.010 | 0.010% |
| Potassium Iodate | 0.005 | 0.005% |
| Glycerol | 5.10 | 5.10% |
| Sodium DiHydrogen | 0.25 | 0.25% |
| Phosphate Dihydrate | | |
| Sodium Hydroxide | 0.85 | 0.85% |
| Ethanol | 0.59 | 0.59% |
| Menthol | 0.01 | 0.010% |
| Benzalkonium Chloride | 0.08 | 0.08% |
| Water | 93.08 | 93.0% |
| | 100.00 | 100.0% |

### Formulation 7:

| Per 100 mL | g | % w/v |
|---|---|---|
| PVP-I | 1.10 | 1.10% |
| Potassium Iodide | 0.010 | 0.010% |
| Potassium Iodate | 0.005 | 0.005% |
| Glycerol | 4.40 | 4.40% |
| Sodium DiHydrogen | 0.15 | 0.15% |
| Phosphate Dihydrate | | |
| Sodium Hydroxide | 0.75 | 0.75% |
| Ethanol | 0.49 | 0.49% |
| Menthol | 0.01 | 0.01% |
| Water | 93.0085 | 93.085% |
| | 100.00 | 100.0% |

### Formulation 8:

| Per 100 mL | g | % w/v |
|---|---|---|
| PVP-I | 0.70 | 0.70% |
| Potassium Iodide | 0.030 | 0.030% |
| Potassium Iodate | 0.015 | 0.015% |
| Glycerol | 4.0 | 4.0% |
| Sodium DiHydrogen | 0.1 | 0.1% |
| Phosphate Dihydrate | | |
| Sodium Hydroxide | 0.45 | 0.45% |
| Ethanol | 0.50 | 0.50% |
| Menthol | 0.10 | 0.10% |
| Water | 93.105 | 94.105% |
| | 100.00 | 100.0% |

### Formulation 9:

| Per 100 mL | g | % w/v |
|---|---|---|
| PVP-I | 0.50 | 0.50% |
| Potassium Iodide | 0.010 | 0.010% |
| Potassium Iodate | 0.005 | 0.005% |
| Glycerol | 5.00 | 5.00% |
| Sodium DiHydrogen | 0.15 | 0.15% |
| Phosphate Dihydrate | | |
| Sodium Hydroxide | 0.75 | 0.75% |
| Ethanol | 0.49 | 0.49% |
| Menthol | 0.01 | 0.010% |
| Water | 93.08 | 93.08% |
| | 100.00 | 100.0% |

### Formulation 10:

| Per 100 mL | g | % w/v |
|---|---|---|
| PVP-I | 0.80 | 0.80% |
| Potassium Iodide | 0.010 | 0.010% |
| Polyethylene Glycol 400 | 4.80 | 4.80% |
| Sodium DiHydrogen Phosphate | 0.15 | 0.15% |
| Sodium Hydroxide | 0.75 | 0.75% |
| Ethanol | 0.49 | 0.49% |
| Menthol | 0.01 | 0.010% |
| Benzalkonium Chloride | 0.1 | 0.10% |
| Water | 92.89 | 92.89% |
| | 100.00 | 100.0% |

### Formulation 11:

| Per 100 mL | g | % w/v |
|---|---|---|
| PVP-I | 0.50 | 0.50% |
| Potassium Iodide | 0.050 | 0.050% |
| Potassium Iodate | 0.005 | 0.005% |
| Glycerol | 5.00 | 5.00% |
| Sodium DiHydrogen | 0.15 | 0.15% |
| Phosphate Dihydrate | | |
| Sodium Hydroxide | 0.7 | 0.7% |
| Ethanol | 0.50 | 0.50% |
| Menthol | 0.01 | 0.010% |
| Water | 93.085 | 93.085% |
| | 100.00 | 100.0% |

### Formulation 12:

| Per 100 mL | g | % w/v |
|---|---|---|
| PVP-I | 1.00 | 1.00% |
| Potassium Iodide | 0.010 | 0.010% |
| Glycerol | 5.50 | 5.50% |
| Sodium Hydrogen Carbonate | 0.15 | 0.15% |
| Ethanol | 0.49 | 0.49% |
| Menthol | 0.01 | 0.010% |
| Water | 92.84 | 92.84% |
| | 100.00 | 100.0% |

### Formulation 13:

| Per 100 mL | g | % w/v |
|---|---|---|
| PVP-I | 1.25 | 1.25% |
| Potassium Iodide | 0.010 | 0.010% |
| Glycerol | 4.60 | 4.40% |
| Citric Acid | 0.325 | 0.325% |
| Sodium Hydroxide | 0.15 | 0.15% |
| Ethanol | 0.22 | 0.22% |
| Menthol | 0.21 | 0.21% |
| Benzalkonium Chloride | 0.25 | 0.25% |
| Water | 92.985 | 92.985% |
| | 100.00 | 100.0% |

### Formulation 14:

| Per 100 mL | g | % w/v |
|---|---|---|
| PVP-I | 0.30% | 0.30% |
| Potassium Iodide | 0.010 | 0.010% |
| Hyaluronic acid | 2.20 | 2.20% |
| Sodium DiHydrogen | 0.35 | 0.35% |
| Phosphate Dihydrate | | |
| Ethanol | 0.49 | 0.49% |
| Menthol | 0.01 | 0.010% |
| Benzalkonium Chloride | 0.08 | 0.08% |
| Water | 96.56 | 96.56% |
| | 100.00 | 100.0% |

### Formulation 15:

| Per 100 mL | g | % w/v |
|---|---|---|
| PVP-I | 0.10 | 0.10% |
| Potassium Iodate | 0.005 | 0.005% |
| Glycerol | 5.10 | 5.10% |
| Citric Acid | 0.25 | 0.25% |
| Sodium Hydroxide | 0.65 | 0.65% |
| Ethanol | 0.59 | 0.59% |
| Menthol | 0.01 | 0.010% |
| Water | 93.295 | 93.295% |
| | 100.00 | 100.0% |

### Formulation 16:

| Per 100 mL | g | % w/v |
|---|---|---|
| PVP-I | 1.10 | 1.10% |
| Potassium Iodide | 0.010 | 0.010% |
| Glycerol | 3.40 | 3.40% |
| Sodium Hydrogen Carbonate | 0.15 | 0.15% |
| Sodium Hydroxide | 0.75 | 0.75% |
| Menthol | 0.01 | 0.01% |
| Water | 94.58 | 94.58% |
| | 100.00 | 100.0% |

### Formulation 17:

| Per 100 mL | g | % w/v |
|---|---|---|
| PVP-I | 0.70 | 0.70% |
| Potassium Iodide | 0.030 | 0.030% |
| Potassium Iodate | 0.015 | 0.015% |
| Hyaluronic acid | 4.0 | 4.0% |
| Citric Acid | 0.1 | 0.1% |
| Sodium Hydroxide | 0.45 | 0.45% |
| Ethanol | 0.30 | 0.30% |
| Menthol | 0.10 | 0.10% |
| Water | 94.305 | 94.305% |
| | 100.00 | 100.0% |

### Formulation 18:

| Per 100 mL | g | % w/v |
|---|---|---|
| PVP-I | 0.50 | 0.50% |
| Potassium Iodide | 0.020 | 0.020% |
| Polyethylene Glycol 1000 | 7.00 | 7.00% |
| Sodium DiHydrogen | 0.15 | 0.15% |
| Phosphate Dihydrate | | |
| Sodium Hydroxide | 0.65 | 0.65% |
| Dimethyl Sulphoxide | 0.49 | 0.49% |
| Menthol | 0.01 | 0.010% |
| Water | 91.18 | 91.18% |
| | 100.00 | 100.0% |

### Biological Examples

### Example 1—Activity Against HRV-14

Human rhinovirus 14 (HRV-14) is known to have a degree of resistance to PVP-I and therefore was selected as a representative “hard to kill” non-enveloped virus for the purposes of discriminating the differential activity of PVP-I formulations. For example, Kawana and colleagues (Kawana, R., et al. Inactivation of Human Viruses by Povidone-Iodine in Comparison to Other Antiseptics. Dermatology 1997, 195:29-35) reported that treatment of HRV-14 with a 1% PVP-I solution using the formulation sold as Betadine™ yielded a reduction in viral titers of only 1.3 and 1.7 log 10, based on a 0.5 and 10 minute exposure, respectively. These times are relevant to proposed use of the present invention because the likely effective residence and activity time for PVP-I solution in the nose is approximately less than 5 minutes, due to mucociliary clearance and inactivation by mucins and other proteinaceous material.

### Method

The Formulation 2 described in the present invention was selected for testing against various commercial PVP-I formulations. Other commercial formulations described below are as disclosed in Niazi, S. K. (2009) Handbook of Pharmaceutical Manufacturing Formulations, Second Edition, Volume Series, 2009. Informa Healthcare USA, Inc., New York, N.Y. 10017. In addition, because published data was available against HRV-14, a 0.45% PVP-I liposomal investigative formulation (reported by Wutzler et al. Antiviral Research 2002, 54:89-97) and a reference 1.0% PVP-I solution in phosphate buffer (reported by Kawana et al. Dermatology 1997, 195:29-35)

HRV-14 virus was exposed to Formulation 2 (0.5% PVP-I) of the present invention for the indicated time period at 34° C. prior to quenching the mixture by 1:10 dilution using ice cold culture medium (DMEM-F12, 2% fetal bovine serum) and measurement of viral titers by TCID₅₀ assay on HeLa cells.

Betaisodona data are as reported by Reimer (2002) (Reimer, R., et al. Antimicrobial Effectiveness of Povidone-Iodine and Consequences for New Application Areas. Dermatology 2002, 204:114-120) and Wutzler (2002), (Wutzler, P., et al. Virucidal activity and cytotoxicity of the liposomal formulation of povidone iodine. Antiviral Research 2002, 54:89-97), respectively. Liposomal PVP-I data are also from Wutzler (2002). Isodine Gargle data and PVP-I solution in phosphate buffer are as reported by Kawana and colleagues (1997).

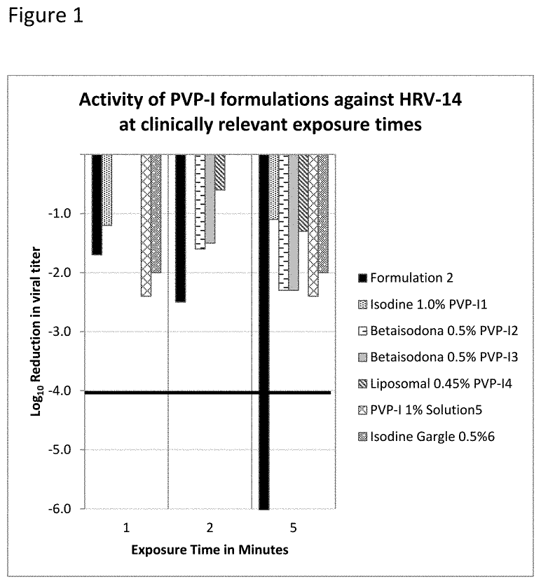

Units are log₁₀ viral titer reduction. Results are shown in FIG. 1. The heavy horizontal line corresponds to a 4 log 10 reduction (corresponding to a 99.99% reduction in viral infectivity) in accordance with the Therapeutic Goods Administration (TGA) guidelines for virucidal testing: TGA instructions for disinfectant testing, Disinfectants, sterilants and sanitary products, Version 2.1, March 2020 (TGA Department of Health, Australian Government).

The 0.5% PVP-I formulation of the present invention (Formulation 2) showed a markedly increased virucidal activity compared to other PVP-I formulations (concentrations between around 0.5% and 1.0%), at clinically-relevant exposure times of 2 and 5 minutes and comparable activity at 1 minute. Notably, Formulation 2 was the only PVP-I formulation which exceeded the 4 log₁₀ reduction for virucidal products with a 5-minute exposure period. The other formulations required at least 15 minutes to achieve this standard.

0.5% PVP-I formulation of the present invention was clearly more effective than the other PVP-I formulations previously disclosed, including 1% PVP-I solutions tested (Isodine Gargle and PVP-I solution in phosphate buffer).

### Example 2—Virucidal Activity of Various Solutions Against SARS-CoV-2

The activity of the example Formulation 2 was compared with a simple solution in water of PVP-I at the same concentration and the background formula of Formulation 2 without the PVP-I.

### Method

Virus, Media, and Cells SARS-CoV-2 virus stocks were prepared by growing virus in Vero 76 cells. Test media used was MEM supplemented with 2% FBS and 50 μg/mL gentamicin.

Tested products were: Test solution 1 (Formulation 2 containing 0.5% PVP-I), Test solution 2 (0.5% PVP-I in water), Test solution 3 (Formulation 2 without PVP-I). The test solutions were tested at full strength at three contact time-points: 15 sec, 5 min, and 15 min to reflect clinically-relevant exposure periods. SARS-CoV-2 virus stock was added to tubes at 1/10 of the test solution, so the final concentrations of solution tested was 90%. The test solutions were added to two tubes of each solution to serve as toxicity controls. Ethanol (70%) was tested in parallel as a positive virucidal control and a virus control was included for each contact time-point.

Solution and virus were incubated at 37° C. for three contact times of 15 seconds, 5 minutes, and 15 minutes. Following contact period, the solutions were neutralized by a 1/10 dilution in test media containing 10% FBS.

Neutralized samples were serially diluted using eight log 10 dilutions in test medium. Each dilution was added to 4 wells of a 96-well plate with 80-100% confluent Vero E6 cells. The toxicity controls were added to an additional 4 wells and 2 of these wells were infected with virus to serve as neutralization controls, ensuring that residual sample in the titer assay plated did not inhibit growth and detection of surviving virus. All plates were incubated at 37° C., 5% CO₂.

On day 6 post-infection plates were scored for presence or absence of viral cytopathic effect (CPE). The Reed-Muench method was used to determine end-point titers (50% cell culture infectious dose, CCID₅₀) of the samples, and the log reduction value (LRV) of the compound compared to the negative (water) control was calculated.

Virus controls were tested in diluent and the reduction of virus in compound-treated test wells compared to virus controls was calculated as the log reduction value (LRV). Toxicity controls were tested to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

### Results

Virus titres and LRV for the three solutions against SARS-CoV-2 are shown in Table 1. Compound toxicity was not observed for any of the solutions. The 70% ethanol positive control was effective and reduced virus titer to a baseline of 0.67 log₁₀ CCID₅₀/ml.

The test solution 1 most consistently reduced virus titer to the greatest extent as compared with the other test solutions and was the only formulation to reach a 4-log reduction within 5 minutes. Solution 1 (Example Formulation 2) had a LRV of 3.5 and 4.0 at the 15 sec and 5 min contact times, and reduced virus below limit of detection after 15 min of contact time (Table 1). PVP-I solution was also intermediately active, reducing virus, but not to the extent of Solution 1 (Table 1). The formula without PVP-I was not active at any of the selected contact points.

Neutralization controls demonstrated that residual sample did not inhibit virus growth and detection in the endpoint titer assays in wells that did not have cytotoxicity. Virus controls and positive controls performed as expected.

TABLE 1
Virucidal efficacy against SARS-CoV-2 after
incubation with virus at 37° C.

| | | Virus | |
|---|---|---|---|
| | Incubation | Titer^{a} | LRV^{b} |
| Test Solution 1 | 15 seconds | 1.5 | 3.5 |
| Test Solution 2 | 15 seconds | 2.5 | 2.5 |
| Test Solution 3 | 15 seconds | 5.0 | 0 |
| Ethanol | 15 seconds | 0.67 | 4.3 |
| Virus Control | 15 seconds | 5.0 | — |
| Test Solution 1 | 5 minutes | 1.0 | 4.0 |
| Test Solution 2 | 5 minutes | 1.67 | 3.3 |
| Test Solution 3 | 5 minutes | 4.5 | 0.5 |
| Ethanol | 5 minutes | <0.67 | >4.3 |
| Virus Control | 5 minutes | 5.0 | — |
| Test Solution 1 | 15 minutes | <0.67 | >4.3 |
| Test Solution 2 | 15 minutes | <0.67 | >4.3 |
| Test Solution 3 | 15 minutes | 5.5 | 0 |
| Ethanol | 15 minutes | <0.67 | >4.3 |
| Virus Control | 15 minutes | 5.3 | — |

^{a}Log₁₀ CCID₅₀ of virus per mL, mean of 3 replicates ± standard deviation.
^{b}LRV (log reduction value) is the reduction of virus compared to the virus control.
Test Solution 1 = Example Formulation 2 supplied in amber glass.
Test Solution 2 = 0.5% PVP-I aqueous solution supplied in amber glass.
Test Solution 3 = Example Formulation 2 without PVP-I.

### Example 3—Activity of Various Solutions Against MRSA

### Method

S. aureus ATCC 33591 was streaked from frozen stock onto Tryptic Soy Agar (TSA) plates and grown overnight at 37° C.

On the day of the assay, the bacteria inocula was prepared by suspending ATCC 33591 colonies in sterile PBS to generate 1.0 McFarland standards in 2 mL volume. The suspension was centrifuged to pellet the bacteria, then resuspended in 0.2 mL sterile PBS. Two independent inocula was prepared using this method.

2 mL of each of four test solutions were dispensed into sterile borosilicate glass tubes; 2 mL of sterile PBS was also prepared to determine inoculum density (i.e. time 0 control). One set of test solutions was prepared and tested for each independent inoculum.

50 μL of each inoculum was added to each test solution or to PBS control.

After the addition of inoculum, each test solution was vortexed on high and a timer was started; at each time point, 20 μL was removed from the inoculated solution and added to 180 μL DE Neutralization buffer. This was then 10-fold serially diluted and 100 μL of the appropriate dilution was plated on TSA plates.

TSA plates were incubated overnight at 37° C., after which CFU was enumerated. The results are shown in Tables 2 and 3.

TABLE 2
MRSA time-kill results
Species S aureus
Strain: ATCC33591

| | | Time point | CFU | Dilution | |
|---|---|---|---|---|---|
| | | (seconds | count | (10\hat{ } X) | CFU/mL |
| Sample 1 | Replicate 1 | 0 | 101 | 5 | 1.01E+08 |
| | | 15 | 30 | 2 | 3.00E+04 |
| | | 30 | 0 | 1 | 1.00E+01 |
| | | 60 | 0 | 1 | 1.00E+01 |
| | Replicate 2 | 0 | 70 | 5 | 7.00E+07 |
| | | 15 | 89 | 1 | 8.90E+03 |
| | | 30 | 0 | 1 | 1.00E+01 |
| | | 60 | 0 | 1 | 1.00E+01 |
| Sample 2 | Replicate 1 | 0 | 101 | 5 | 1.01E+08 |
| | | 15 | 0 | 1 | 1.00E+01 |
| | | 30 | 0 | 1 | 1.00E+01 |
| | | 60 | 0 | 1 | 1.00E+01 |
| | Replicate 2 | 0 | 70 | 5 | 7.00E+07 |
| | | 15 | 0 | 1 | 1.00E+01 |
| | | 30 | 0 | 1 | 1.00E+01 |
| | | 60 | 0 | 1 | 1.00E+01 |
| Sample 3 | Replicate 1 | 0 | 101 | 5 | 1.01E+08 |
| | | 15 | TNTC | 3 | TNTC |
| | | 30 | TNTC | 3 | TNTC |
| | | 60 | TNTC | 3 | TNTC |
| | Replicate 2 | 0 | 70 | 5 | 7.00E+07 |
| | | 15 | TNTC | 3 | TNTC |
| | | 30 | TNTC | 3 | TNTC |
| | | 60 | TNTC | 3 | TNTC |

1.00E+01 indicate no CFU were recovered at the lowest dilution plated (that is 10⁻¹); the data value shown is the limit of detection for the assay (i.e. 10 CFU/mL).
TNTC = too numerous to count.

TABLE 3
MRSA time-kill results: average CFU/mL

| Time | | | |
|---|---|---|---|
| point | Average CFU/mL | | |
| (sec) | Sample 1 | Sample 2 | Sample 3 |
| 0 | 8.55E+07 | 8.55E+07 | 8.55E+07 |
| 15 | 1.95E+04 | 10 | TNTC |
| 30 | 10 | 10 | TNTC |
| 60 | 10 | 10 | TNTC |

TNTC = too numerous to count.
Sample 1 = Formulation 2 supplied in amber glass.
Sample 2 = 0.5% PVP-I/H₂O supplied in amber glass.
Sample 3 = Formulation 2 without PVP-I supplied in amber glass

The results indicated that the example Formulation 2 was not more active than PVP-I solution in water against bacteria, demonstrating that the surprisingly enhanced activity against viruses is not seen with bacteria.

1. An aqueous virucidal formulation, comprising:
(a) about 0.10% to about 1.25% w/v PVP-I;
(b) 0.00% to about 15% w/v of hyaluronic acid, polyethylene glycol and/or glycerol;
(c) 0.00% to about 0.20% w/v of menthol;
(d) 0.00% to about 30% w/v of a polar solvent;
(e) 0.00% to about 5% w/v of sodium hydrogen phosphate or similar buffer; and
(f) 0.00% to about 0.20% w/v of iodide and/or iodate salt;
wherein the formulation comprises at least two of (b)-(f), and
wherein % w/v is based on % w/v of the total formulation.

2. An aqueous virucidal formulation according to claim 1, comprising:
(a) about 0.10% to about 1.25% w/v PVP-I;
(b) about 0.005% to about 0.20% w/v of iodide and/or iodate salt; and
(c) about 0.005% to about 0.05% w/v menthol.

3. An aqueous virucidal formulation according to claim 1, comprising:
(a) about 0.10% to about 1.25% w/v PVP-I;
(b) about 0.005% to about 0.01% w/v of an iodate salt;
(c) about 0.005% to about 0.05% w/v menthol;
(d) about 0.005% to about 0.10% w/v of an iodide salt; and
(e) about 1.0% w/v to about 15% w/v of glycerol.

4. An aqueous virucidal formulation according to claim 1, comprising:
(a) about 0.10% to about 1.25% w/v PVP-I;
(b) about 0.005% to about 0.01% w/v of an iodate salt;
(c) about 0.005% to about 0.05% w/v menthol;
(d) about 0.005% to about 0.10% w/v of an iodide salt;
(e) about 1.0% w/v to about 15% w/v of glycerol; and
(f) about 0.2% w/v to about 30% w/v of a polar solvent other than water.

5. An aqueous virucidal formulation according to claim 1, comprising:
(a) about 0.10% to about 1.0% w/v PVP-I;
(b) about 0.005% to about 0.01% w/v of an iodate salt;
(c) about 0.005% to about 0.05% w/v menthol;
(d) about 0.005% to about 0.10% w/v of an iodide salt;
(e) about 1.0% w/v to about 10% w/v of glycerol;
(f) about 0.2% w/v to about 1.0% w/v of a polar solvent other than water;
(g) about 0.05% to about 0.20% w/v of sodium dihydrogen phosphate dihydrate;
(h) sodium hydroxide q.s.; and
(i) water q.s.

6. An aqueous virucidal formulation according to claim 1, wherein the concentration of PVP-I is about 0.25% to about 1.00% w/v.

7. An aqueous virucidal formulation according to claim 5, wherein the concentration of PVP-I is about 0.50% w/v.

8. An aqueous virucidal formulation according to claim 5, wherein the polar solvent other than water is ethanol.

9. An aqueous virucidal formulation according to claim 1, wherein the pH of the formulation is about 3 to about 6.

10. A method of treating viral infection in a subject comprising administering to the subject an effective amount of the aqueous virucidal formulation according to claim 1.

11. A method according to claim 10, wherein the formulation is administered intranasally.

12. A method of reducing the volume of mucous secreted, or reducing the activity, viability or number of viruses contained in secreted mucous, or reducing the period during which virus-laden mucous is present, and thereby reducing the risk of viruses migrating to secondary sites in the respiratory tract to establish or contribute to secondary illnesses, including the step of administering to a subject an effective amount of an aqueous virucidal formulation according to claim 1.

13. The method according to claim 12, wherein the secondary illness is a respiratory tract infection.

14. The method according to claim 13, wherein the secondary illness selected from the group selected from sinusitis, bronchitis, and otitis media.

15. A method according to claim 10, wherein the aqueous virucidal formulation is administered nasally at a temperature of between 10° to 35° C.
